(19) United States
(12) Patent Application Publication
(10) Pub. No.: US 2012/0245527 A1
(43) Pub. Date: Sep. 27, 2012

(54) INTRODUCER VALVE

(75) Inventors: David Wesley Stephens, Dallas, TX (US); Joseph Allan Pepin, Rowlett, TX (US)

(73) Assignee: GALT MEDICAL, Garland, TX (US)

(21) Appl. No.: 13/429,519

(22) Filed: Mar. 26, 2012

Related U.S. Application Data
(60) Provisional application No. 61/467,847, filed on Mar. 25, 2011.

(51) Int. Cl. A61B 17/34

(52) U.S. Cl. 604/167.03

(57) ABSTRACT

The present invention is directed to a novel valve for an introducer and a method for using the introducer valve to facilitate the insertion of one or more medical devices into a patient's body. The introducer valve of the present invention includes two or more valves seals adapted to form a seal around medical devices having a wide range of sizes. In one embodiment, the valve includes a longitudinal passageway for receiving a medical device, a first seal forming a seal across longitudinal passageway, and an annular second seal, wherein an aperture of the second seal is defined by a semi-toroidal portion that forms a curved inner surface that forms a seal with the medical device.

### BACKGROUND OF THE INVENTION

1. Field of the Invention

The present invention generally relates to valves for use with an introducer. In particular, the invention is directed to a hemostatic valve that facilitates vascular access.

2. Description of the Related Technology

Introducers that facilitate the insertion of a catheter into a blood vessel are well known in the art. Some introducers require physicians place their thumbs over the lumen of the introducer to form a temporary seal, thereby minimizing the occurrence of air embolisms or blood leakage. In a small number of these cases, however, air embolisms still arise, requiring further intervention and occasionally resulting in death. Additionally, despite the aforementioned manual sealing technique, blood leakage also occurs when using such introducers, posing a safety hazard to physicians.

More recently, introducer assemblies have been developed that include valve structures which seal against medical devices inserted therein. Although the valves may sealably engage a medical device of a specific size, they are unable to form a seal with different sizes of medical devices. For example, conventional introducer valves capable of forming a seal with a 0.018 inch guidewire are generally unable to form a seal with a 10 FR catheter. This means that different valves would have to be used for introduction of each differently sized device.

In an attempt to enhance sealing capability, some introducers have two or more valve elements. These valve elements, however, still suffer drawbacks when used with medical devices of varying sizes. U.S. Pat. No. 6,712,791, for example, teaches a splittable medical valve assembly including two or more sealing elements adhesively affixed within a valve body, in, for example, FIGS. 3 and 6-8. The number and configuration of its sealing elements is dictated by, and specific for, the selected medical device to be inserted within the valve. One sealing element is configured as a thin seal with a slit formed therein to seal with, for example, a guidewire, while a second sealing element is constructed as a disk shaped seal insert. The second sealing element has an inner surface that defines an aperture permitting passage of a medical device. A drawback of this device is that the second sealing element has limited flexibility due to the fact that it is formed from a solid ring. Thus, it is not adapted to receive medical devices having a diameter that is somewhat larger than the circumference of the aperture. Also, the structure of the second sealing element, namely the straight walled inner wall shown in FIG. 9, creates a large area of frictional contact and resistance between the inner wall of the sealing element and the medical device. Forcing a medical device slightly larger than the aperture into such an aperture would therefore meet with significant resistive force due to both the limited flexibility of the sealing element and the relatively large area of frictional engagement between the device and the inner wall of the seal.

In view of the deficiencies of the prior art, there is a need to develop an improved introducer valve capable of forming a seal with a variety of medical devices having a wide range of sizes.

### SUMMARY OF THE INVENTION

In a first aspect, the present invention relates to an introducer valve capable of being coupled to an introducer. The valve body includes a longitudinal passageway for receiving a medical device; a first seal across the longitudinal passageway including a slit therein; and a second seal spaced apart from the first seal, wherein the second seal comprises an inner surface of a semi-toroid forming an aperture that is capable of sealing with a medical device inserted therein.

In a second aspect, the present invention relates to an introducer valve having a valve body capable of being coupled to an introducer. The valve body includes a longitudinal passageway for receiving a medical device; a first seal across the longitudinal passageway including a slit therein; and a second seal spaced apart from the first seal. The second seal includes an inner surface forming a first aperture through the second seal and wherein a flexible region of the second seal forming the inner surface has a tensile elongation of 190-220. Preferably, the materials used to fabricate the seals of the present invention can deflect and stretch by second order, non-linear deformation.

In a third aspect, the present invention relates to an introducer valve capable of being coupled to an introducer. The valve body includes a longitudinal passageway for receiving a medical device; a first seal across the longitudinal passageway including a slit therein; a semi-toroidal second seal spaced apart from the first seal, wherein the semi-toroidal second seal comprises a first aperture. Said aperture can be, for example, flat, or conical in shape.

### BRIEF DESCRIPTION OF THE DRAWINGS

FIG. 1(a) is a perspective view showing half of a first two part valve embodiment of the present invention.

FIG. 1(b) is a side view of the half of the valve of FIG. 1(a).

FIG. 1(c) is a perspective view showing the half of the valve of FIG. 1(a) positioned within a split introducer, shown in a perspective cross-sectional view.

FIG. 1(d) is a side view of the half of the valve of FIG. 1(a) positioned within a split introducer.

FIG. 2(a) is a perspective view showing half of a second embodiment of a two part valve of the present invention.

FIG. 2(b) is side view of the half of the valve of FIG. 2(a).

FIG. 3(a) is a perspective view showing half of a third embodiment of a two part valve of the present invention.

FIG. 3(b) is side view of the half of the valve of FIG. 3(a).

FIG. 4(a) is an exploded view illustrating the assembly of one embodiment of valve in accordance with the present invention within a splittable introducer.

FIG. 4(b) is a perspective view of the assembled splittable introducer of FIG. 4(a).

FIG. 5(a) is a perspective view showing half of one preferred embodiment two part valve embodiment of the present invention.

FIG. 5(b) is a side view of the half of the valve of FIG. 5(a).

FIG. 5(c) is a side view of the half of the valve of FIG. 5(a) with the location of adhesive application points indicated.

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

As used herein, “medical device” refers to any medical instrument intended for placement within a body. Exemplary medical devices may include, but are not limited to, cannulas, catheters, dilators, guidewires, needles, syringes, probes, trocars, robotic actuation arms, or imaging devices, such as video cameras.

As used herein, “score line” refers to any cut, fissure, thinner portion or groove that partially or completely penetrates through a surface or material to facilitate separation along the cut, fissure or groove.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

The present invention is directed to a novel introducer valve and a method for using the introducer valve to facilitate the insertion of one or more medical devices into a patient's body. The invention provides an introducer valve having two or more valve seals that are capable of forming seals with medical devices of different sizes. Embodiments of the invention also provide relatively flexible valve seals for reducing resistance to insertion of medical devices therein. Embodiments of the invention also provide valve seals with an inner surface of a semi-toroid to enhance the flexibility of the valve seal, increase the sealing surface area of the valve seal and/or reduce frictional engagement between the valve seal and a medical device during insertion of a medical device therein.

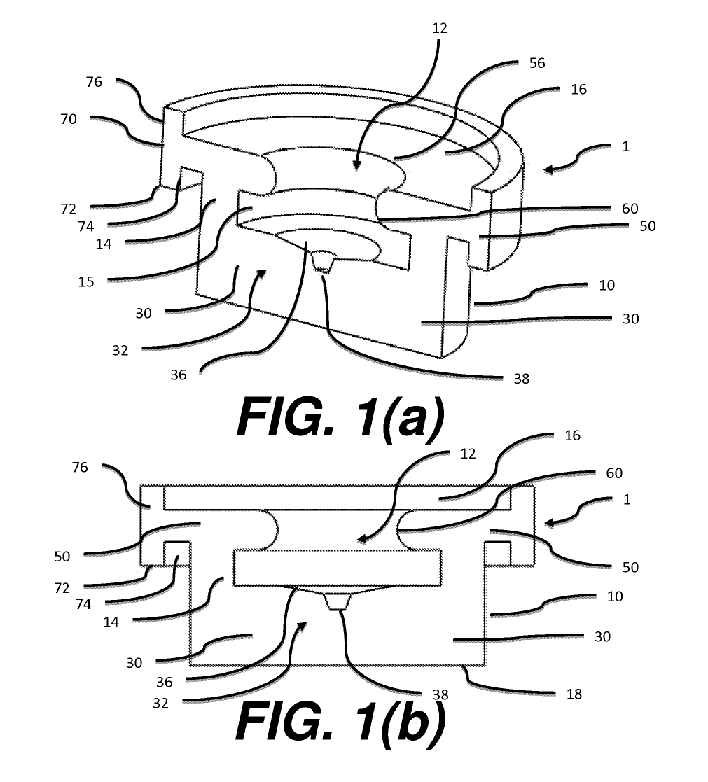

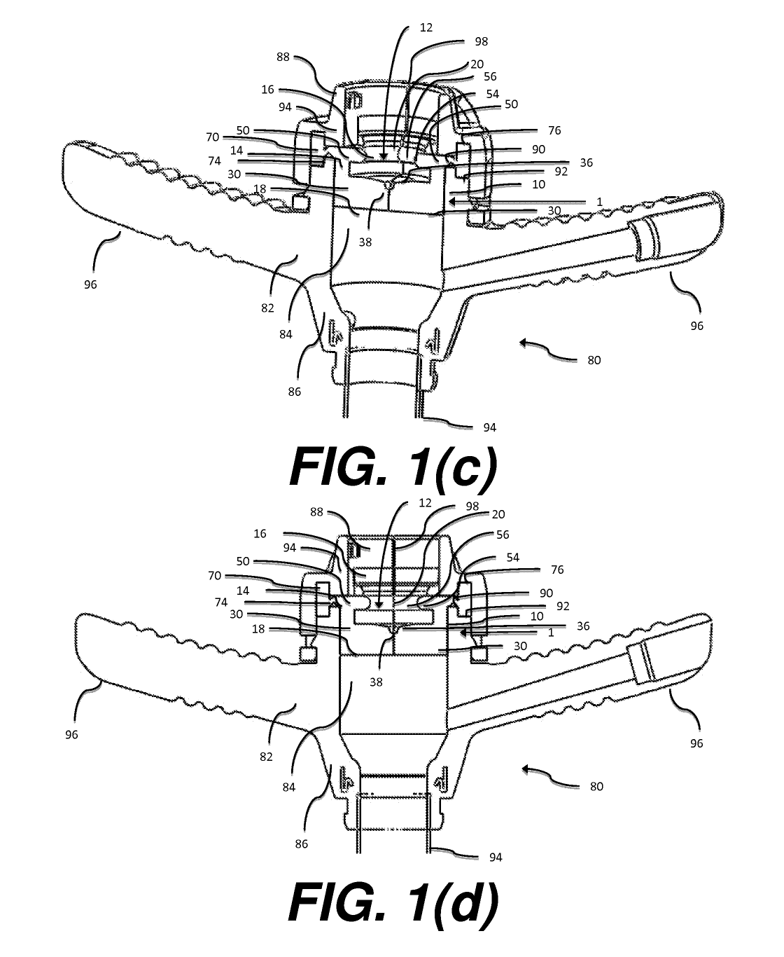

Referring now to the drawings, wherein like reference numerals designate corresponding structures throughout the several views, and referring in particular to the first exemplary embodiment of FIGS. 1(a)-1(d), the introducer valve of the present invention is formed from two halves 1 of the introducer valve, only one of which halves 1 is shown in these figures. The introducer valve includes two or more seals 30, 50, each adapted to form a seal around medical devices having a wide range of sizes. As shown, the introducer valve has a valve body 10 formed from two halves 1 which define a longitudinal passageway 12 for receiving a medical device. Along longitudinal passageway is provided a first seal 30 that forms a seal across longitudinal passageway 12 when no medical device is inserted within longitudinal passageway 12. First seal 30 is formed by contact between surfaces of respective halves 1 of the introducer valve. First seal 30 is also capable of a sealing engagement with a medical device having a relatively small cross-sectional area, such as a guidewire.

The introducer valve also includes a second seal 50 capable of forming an interference seal with a medical device having a relatively larger cross-sectional area than devices for which first seal 30 is adapted. Second seal 50 has a semi-toroidal shape defining an inner surface 54 that is capable of sealably engaging a suitably sized medical device which may be tailored for a range of suitably sized devices. For example, devices of from 3 French to 12 French (0.035″-0.165″) may be sealed with only one size seal of, for example, 0.030″. Optionally, the introducer valve may further include one or more supplemental seals 60 each having a semi-toroidal shape defining an inner surface 66 adapted to form a seal with a medical device having a suitably sized cross-sectional area. In an exemplary embodiment, valve body 10 includes a plurality of flexible, annular seals having apertures of different sizes adapted for sealing engagement with a wide range of sizes of medical devices.

Valve body 10 may have any shape, size or configuration suitable for forming a seal around two or more medical devices of different sizes and adapted to be coupled with an introducer 80. As shown in FIG. 1(a), valve body 10 includes a wall 14 for defining longitudinal passageway 12. Optionally, valve body 10 may further include a coupling member 70 for coupling valve body 10 to a suitably shaped introducer 80. Preferably, valve body 10 has a substantially cylindrical or conical structure. The proximal entrance of longitudinal passageway 12 is defined by an opening in a proximal end 16 of valve body 10. The distal exit of longitudinal passageway 12 is formed at a distal end 18 of valve body 10 when a medical device is passed through first seal 30, and typically communicates with an introducer sheath 94 of introducer 80. To better effect this seal, the surface 30 may have an adhesive applied to the surface, bonding the two halves together across the aperture. The adhesive can be of similar compliance as the valve material. Examples include but are not limited to silicone, polyurethane, or other elastomers.

Longitudinal passageway 12 is defined by an interior surface 15 of wall 14 and passes through two or more seals 30, 50 and 60. In the embodiment shown in FIG. 1(a), longitudinal passageway 12 has a generally cylindrical configuration defined by interior surface 15 of wall 14 extending between first seal 30 and second seal 50.

A first seal 30 of valve body 10 operates to providing a sealing engagement with medical devices having a small cross-sectional area, such as small gauge needles or guidewires. As shown in FIGS. 1(a), 2(a) and 3(a), first seal 30 is formed by interior surface 15 of cylindrical wall 14 along a pair of mating surfaces 32 and is generally located proximate to distal end 18 of valve body 10. First seal 30 is formed by a pair of continuous, self-sealing mating surfaces 32 that form a barrier across longitudinal passageway 12 which prevents passage of fluid and gas when no medical device is inserted within the introducer valve. Mating surfaces 32 form a seal by virtue of the fact that they are pressure fitted together as a result of pressure exerted by hub 82 of introducer 80 on an outer surface of valve body 10. Upon positioning a suitably sized medical device within first seal 30, first seal 30 opens along mating surfaces 32 to allow passage of the medical device therethrough and forms a seal around the medical device to prevent passage of fluids and gases therethrough. When valve body 10 and first seal 30 are constructed as an assembly of two or more component parts that are pressure fitted together, the two or more parts of first seal 30 form an interference fit that creates a seal across longitudinal passageway 12. A medical device, however, may be inserted through the junction between and form a seal with the two or more parts of first seal 30. Preferably, first seal 30 is capable of providing a sealing engagement with a medical device having an effective diameter of from about 0.010″ to about 0.050″, preferably, about 0.005″ to about 0.075″, and more preferably, about 0″ to about 0.160″. As the cross-sectional area increases the larger the effective diameter that can be sealed, but as the cross-sectional area increases so does the insertion force unless there is a significant drop in durometer. A balance is needed between seal 30 and seal 50 and/or 60.

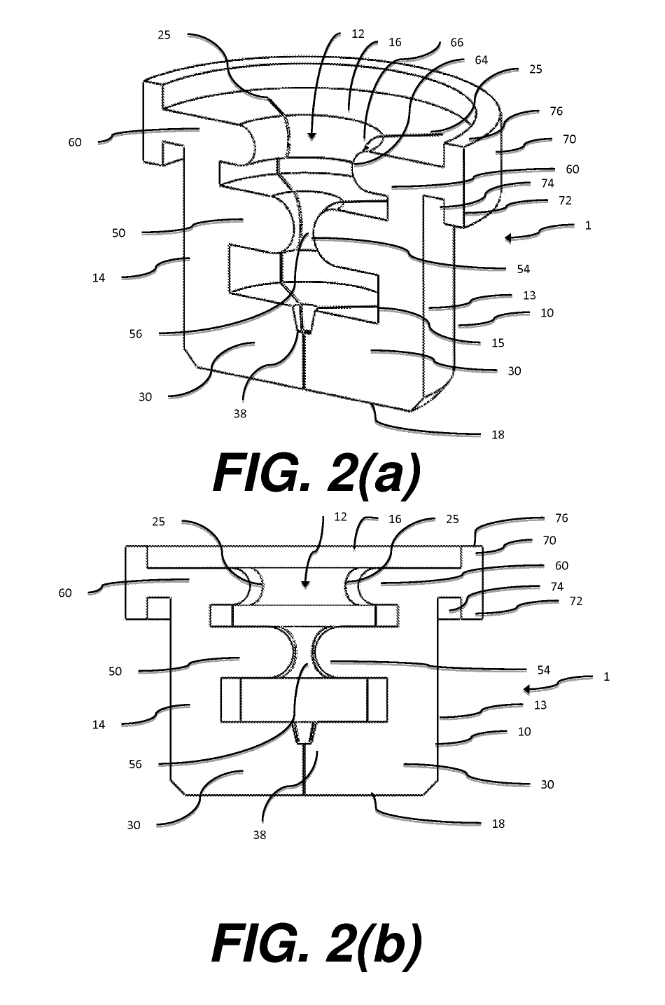

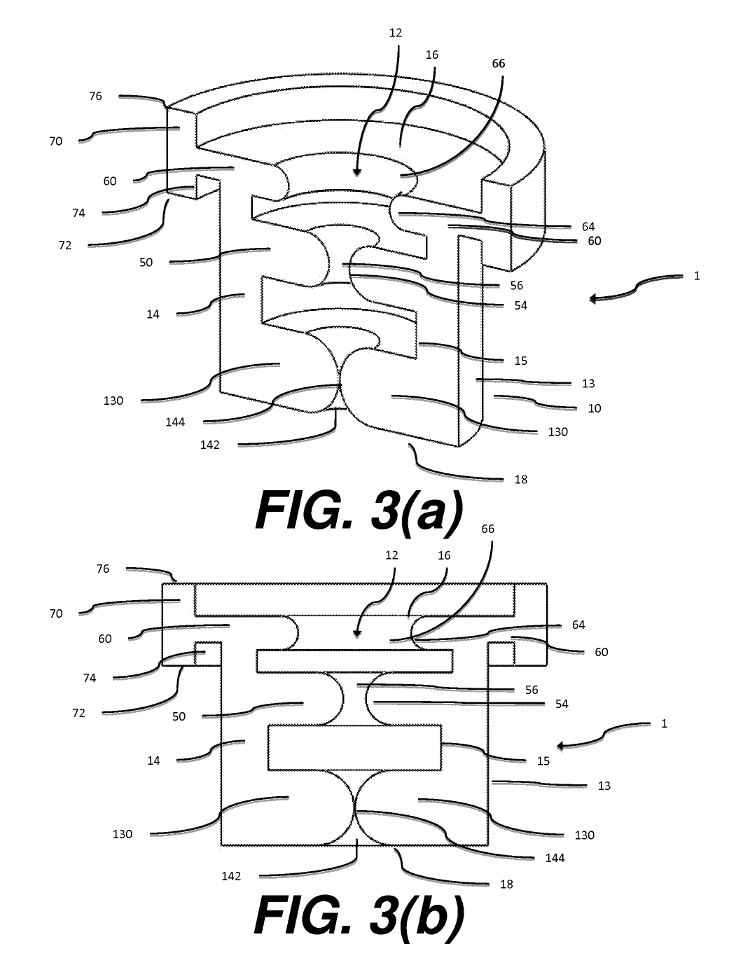

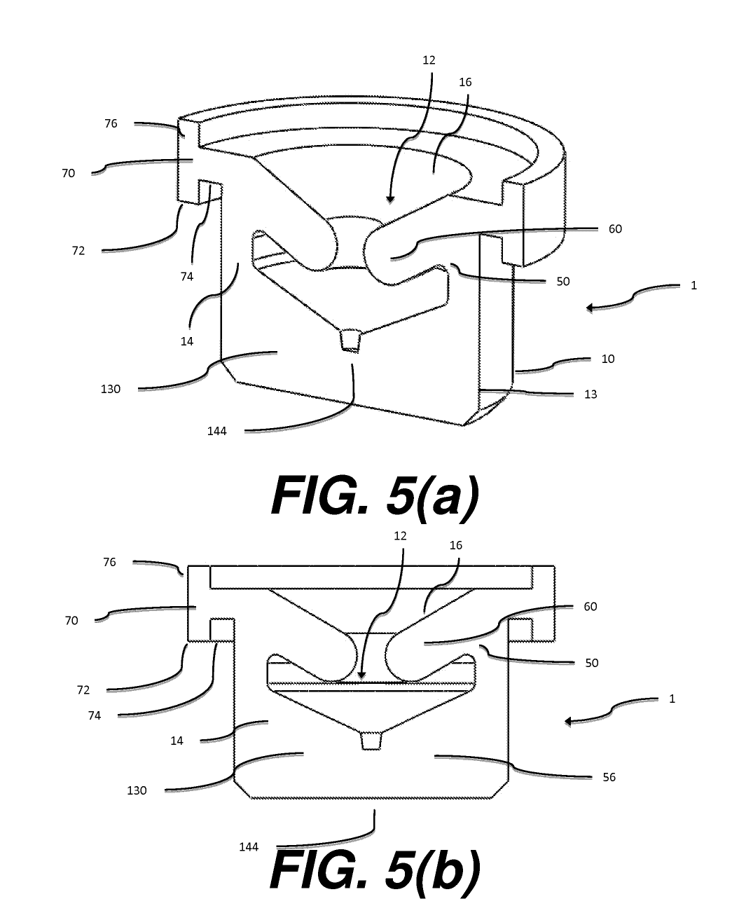

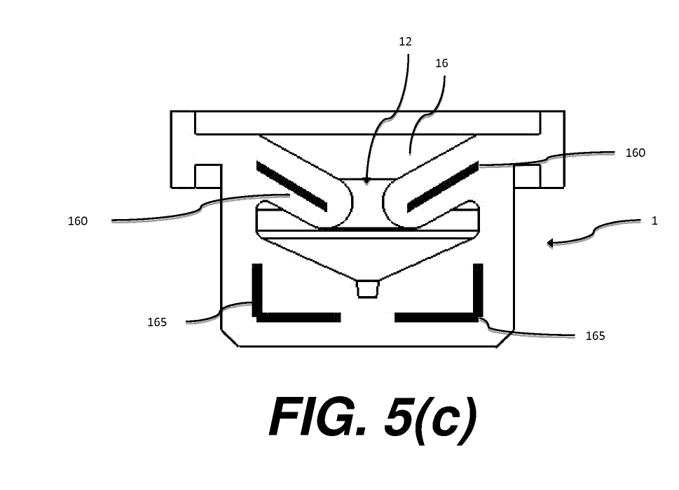

In the embodiments shown in FIGS. 1(a)-2(b), first seal 30 is formed by two mating surfaces 32 that are pressure fitted together along a plane 20, as shown in FIGS. 1(c)-1(d). The first seal 30 may have a depression 36 aligned with longitudinal passageway 12 and spanning the first and second halves 1 of valve body 10, as shown in FIGS. 1(a)-1(d). Depression 36 preferably has a tapered configuration formed in the upper surface of first seal 30 with a maximum depth at its center that accommodates different types and forces of engagement of different sized and shaped medical devices. When depression 36 is tapered, the depth of depression 36 gradually decreases in a distal direction along longitudinal passageway 12. This tapered configuration facilitates the insertion of larger medical devices, into and through first seal 30 by guiding the distal end of the medical device into alignment with plane 20 in which mating surfaces 32 contact one another and increasing the flexibility of the material forming first seal 30. Preferably, depression 36 has a frustum of a cone, conical, semispherical or pyramidal shape. In an alternative embodiment shown in FIGS. 2(a)-2(b) and 3(a)-3(b), first seal 30 may have a substantially planar upper surface oriented substantially perpendicular to wall 14 with no depression 36 therein. In the embodiment of FIGS. 5(a)-5(b), first seal 30 may have a substantially conical upper surface oriented angularly to the wall 14 with a dispersion 16 therein.

As shown in FIGS. 1(a)-3(b) and 5(a)-5(b), optionally, first seal 30 may further have an indentation 38 adapted to facilitate insertion of small medical devices, or medical devices with small tips, into first seal 30. Indentation 38 preferably has a cylindrical, conical, semicircular or pyramidal configuration. Indentation 38 is aligned with the plane 20 in which mating surfaces 32 contact one another to ensure alignment of inserted medical devices with plane 20 to facilitate their insertion through first seal 30. In embodiments including a depression 36, as shown in FIGS. 1(a)-1(d) and 5(a)-5(b), indentation 38 is preferably located in a central region of depression 36.

In another embodiment shown in FIGS. 3(a)-3(b), first seal 130 is configured in a similar manner to second and supplemental seals 50, 60, described below, but having no aperture. In this embodiment, annular first seal 130 has a body including a first section proximal to wall 14 and an inner surface 144 formed by a portion having a semi-toroidal configuration for engaging a medical device as it is positioned through first seal 130. The first section of first seal 130 has an upper surface and parallel lower surface, both of which protrude from wall 14 at an angle of about 75° to about 95°, preferably, about 80° to about 95°, and more preferably, about 85° to about 90°. In an exemplary embodiment, the first section, protrudes from wall 14 in a direction substantially perpendicular to wall 14. The portion having a semi-toroidal configuration or a combination of the first section and the portion having a semi-toroidal configuration may be substantially cylindrical, elliptical, or parabolic.

In one embodiment, integrally formed with the first section and spaced apart from wall 14 is a semi-toroidal portion defining an inner surface 144. First section may be substantially cylindrical as shown in the figures. In various embodiments, the inner surface 144 may have a semi-circular, elliptical, conical, or parabolic profile. As shown, the inner surface 144 contacts at a point 142, forming a continuous, seal across longitudinal passageway 12. For purposes of the present invention, a “semi-toroidal” shape encompasses any configuration with a profile selected from semi-circular, semi-elliptical, semi-conical, or parabolic as well as all configurations therebetween. Preferably, inner surface 144 has a substantially rounded or otherwise curved shape of a surface of a semicircle, conical, ellipse or parabola. The shape of the outer surface of first seal 130, which is connected to wall 14 is not critical to and may be any desired shape.

In another embodiment, there is no separate first section and thus the first seal 130 itself has a semi-circular, semi-elliptical, semi-conical or parabolic profile and extends directly from wall 14.

In this embodiment, a medical device of suitable diameter is inserted through first seal 130 at point 142. During insertion, the region of first seal 130 proximate to point 142 will deflect in a distal direction and a seal will be formed between the medical device and a portion of the upper half of the inner surface 144 as a result of the distal deflection of first seal 130 by the medical device passing therethrough. This configuration of first seal 130 has a low sectional modulus that reduces resistance as a medical device is pushed through first seal 130 relative to, for example, a seal having a conical, cylindrical or frusto-conical profile. In an exemplary embodiment, the ratio of the length of the first section to the length of the semi-toroidal portion is from about 1:1 to about 3:1, more preferably, from about 2:1 to about 3:1 to ensure the desired flexibility of first seal 130.

First seal 30, 130 is sized and is sufficiently flexible to facilitate deflection by a medical device in a distal direction and permit penetration of first seal 30, 130 with minimal resistance to thereby enable the formation of an interference seal with a medical device. In one embodiment, an area of first seal 130 proximate to inner surface 144 has a thickness of about 0.5 mm to about 5 mm, preferably, about 1 mm to about 5 mm, more preferably, about 1.5 mm to about 4 mm, and most preferably, about 1.5 mm to about 2.5 mm. In another embodiment, the surfaces are bonded together as part of the manufacturing process using an adhesive. The optimal adhesive is strong enough to enhance the seal, but weak enough to split during the process of splitting the tearaway hub. The adhesive can have similar mechanical properties as the valve material, such as modulus of elasticity and/or durometer. Examples include but are not limited to silicone, polyurethane, or other elastomers.

Valve body 10 is preferably constructed from two halves 1 as shown in the figures. However, it is also possible to construct valve body 10 from two parts that are unequal in size, though this arrangement will require that longitudinal passage 12 be off-center in valve body 10 or that at least one slit be provided at a suitable location in valve body 10 to form first seal 30. As a result, embodiments employing two unequally sized parts are less preferred.

Valve body 10 can also be formed from three or more parts which would preferably be of equal size to provide a centered longitudinal passage 12. Again, it is possible to form valve body 10 from unequally sized parts, but this is less preferred due to the concerns mentioned above. The formation of valve body 10 from three or more parts is also a less preferably embodiment since it complicates fabrication and construction of the valve and also results in mating surfaces between the parts which would not be positioned in useful locations for splitting the valve for removal.

Valve body 10 may also be formed as a single integral unit, in which case at least one slit 25 would be provided in plane 20 of valve body 10 to form first seal 30. Such a slit may have any configuration and may extend across a portion of, or the entire valve body 10.

In one embodiment, a single linear slit is provided that extends across the entire valve body 10. Alternatively, two or more aligned slits each extending across a portion of valve body 10 may be provided to form first seal 30. Alternatively, two or more intersecting slits 25, optionally arranged in an X-shaped pattern, may be formed in valve body 10 so as to define a plurality of valve leaflets that seal around a medical device placed therethrough, as shown in FIGS. 2(a)-2(b).

As shown in FIGS. 1(a)-1(b), 2(a)-2(d), 3(a)-3(b) and 5(a)-5(b), valve body 10 may further include a second seal 50 adapted for receiving a medical device having a larger cross-sectional area than medical devices for which first seal 30 is adapted. Second seal 50 traverses a portion of longitudinal passageway 12 and is positioned proximal to and spaced from first seal 30 so that a medical device inserted via the proximal end of longitudinal passage 12 encounters second seal 50 prior to encountering first seal 30. As best shown in FIG. 1(d), second seal 50 has a body including a first section attached to wall 14 and semi-toroidal portion defining an inner surface 54 for engaging a medical device as it is positioned through second seal 50. The first section of second seal 50 has an upper surface and parallel lower surface, both of which protrude from wall 14 at an angle of about 75° to about 95°, preferably, about 80° to about 95°, and more preferably, about 85° to about 90°. Second seal 50 may have any configuration or shape as is described above with respect to first seal 130.

By virtue of its smaller cross-section, relative to the rectangular and cylindrical sealing membrane configurations of the prior art, the semi-toroidal profile which defines the curved inner surface 54 increases the flexibility of second seal 50 by reducing the amount of material that must be pushed out of the way by the medical device passing through second seal 50. This configuration of second seal 50 has a low sectional modulus that minimizes resistance as a medical devices is pushed through second seal 50. In an exemplary embodiment, a flexible region of second seal 50, has a tensile elongation of about 190 to about 220. The structural configuration, relatively low sectional modulus, tensile elongation and/or combinations thereof of second seal 50 enable the insertion of medical devices having a diameter that is substantially larger than the aperture positioned through second seal 50, e.g. up to about 20% larger.

Aperture 56 of second seal 50 is sized to provide a sealing engagement with a medical device having a cross-sectional area that is equal to or greater in diameter than aperture 56. In an exemplary embodiment, aperture 56 has a diameter of about 0.25 mm to about 4 mm, preferably, about 0.25 mm to about 3 mm, more preferably, about 0.5 mm to about 2 mm and most preferably, about 0.6 mm to about 0.9 mm

When a medical device having a slightly larger diameter than that of aperture 56 is inserted within aperture 56, the semi-toroidal region of second seal 50 which defines aperture 56 will be deflected in a distal direction and a portion of the upper half of inner surface 54 will provide a sealing engagement with the medical device. As a result of this deflection and the resultant sealing with the medical device along a point on the upper half of inner surface 54, second seal 50 is adapted to accommodate a larger variation in diameters of medical devices and conventional seals having a flat inner surface formed by a rectangular profile. Second seal 50 provides a barrier for preventing passage of gases or liquids through valve body 10 when a medical device of suitable diameter is located therein.

In an exemplary embodiment, second seal 50, specifically at least a region of second seal 50 proximate to inner surface 54, has a thickness of from about 0.5 mm to about 5 mm, preferably, from about 0.75 mm to about 3 mm, more preferably, from about 1 mm to about 2.5 mm, and most preferably, from about 1.5 mm to about 2 mm. Additionally, semi-toroidal second seal 50 is more flexible than a similar seal provided by a flat inner surface of a rectangular profile.

Another advantage of semi-toroidal second seal 50 is that it presents a smaller surface area for direct contact with a medical device passing through second seal 50 than a comparable seal having a flat inner surface formed by a rectangular profile. This has the effect of reducing the frictional engagement between inner surface 64 of second seal 60 and the medical device, relative to the amount of frictional engagement between a flat inner surface formed by a rectangular profile and the same medical device.

Another advantage of a semi-toroidal second seal 50 is that it presents a more tangential contact with a medical device passing through providing a more conforming seal around the device while having lower frictional forces. Said surface could be constructed as a flat surface, or preferably as a conical surface to more preferentially guide target devices into the valve.

As shown in FIGS. 2(a)-5(b), valve body 10 may further include one or more supplemental seals 60 positioned across a portion of longitudinal passageway 12 proximal to first and second seals 30, 50. Supplemental seals 60 are adapted to provide a sealing engagement with a medical device having a diameter or cross-sectional area too large for proper sealing using either of first and second seals 30, 50. Supplemental seals 60 may each have the same type of configuration as described above for second seal 50. A seal with a suitably sized medical device is formed using inner surface 64 of supplemental seal 60. With the exception of the size of the aperture 66 of supplemental seal 60, supplemental seal 60 may have the same shape and configuration as second seal 50. The seals 30, 50, 60 and 130 described above are shown in one preferred order in the drawings but may be implemented in any order, e.g. any of the seals 30, 50, 60 and 130 may be the proximal or distal seal or be located at any location between two or more other seals 30, 50, 60 and 130. In one embodiment, valve body 10 may have a supplemental seal with an aperture having a diameter of about 1.5 mm to about 10 mm, preferably, about 2 mm to about 9 mm, and more preferably, about 3 mm to about 6 mm.

In one embodiment, each aperture 56, 66 may be customized to form a secure seal with a medical device such that the diameter of aperture 56, 66 is equal to or less than the diameter of the medical device by about 5% to about 60%, more preferably, about 15% to about 50%, most preferably, about 30% to about 40%

As best shown in FIGS. 2(a), 3(a) and 5(a), longitudinal passageway 12 has a stepped, configuration suited to forming a seal with a wide range of medical devices at different locations along longitudinal passageway 12 by virtue of first, second and supplemental seals 30, 50, 60. In an exemplary embodiment, valve body 10 may have a plurality of seals 30, 50, 60 having apertures of varying size, preferably, about two to about eight seals, more preferably, about two to about six seals, and most preferably, about two to about four seals.

As shown in FIGS. 2(a) and 3(a), seals 30, 50 and 60 are ordered according to aperture size. Alternatively, the order of seals 30, 50 and 60 may also be inverted such that seal 30 is positioned proximal to the entrance of passageway 12, and seal 60 having the largest aperture is positioned proximal to an outlet of passageway 12. Seal 50 having an intermediate aperture is positioned between seals 30, 60. In this embodiment, flange 70 is positioned proximal to the entrance of the introducer valve. In another embodiment, seal 60 having the largest aperture can be positioned between seal 50 and seal 30. Seals 30, 50 and 60 may also be positioned along passageway 12 such that they are ordered according to material thickness, flexibility and/or durometer hardness, if desired. In an exemplary embodiment, seals 30, 50 and 60 may have different thicknesses, flexibility and durometer hardness that are progressively ordered along longitudinal passageway 12. The ordering of seals 30, 50, 60 in a series along longitudinal passageway 12 in order of decreasing aperture size, decreasing flexural modulus, decreasing durometer hardness, increasing thickness or combinations thereof may be particularly effective in accommodating and forming an interference seal with medical devices of varying sizes.

First seal 30, second seal 50 and one or more supplemental seals 60 are vertically spaced apart from one another to enable the seals 30, 50, 60 to freely and independently deflect when a medical device is passed through longitudinal passageway 12. In an exemplary embodiment, the distance between two adjacent seals 30, 50, 60 is from about 0.25 mm to about 3 mm, preferably, from about 0.25 mm to about 2 mm, and more preferably, from about 0.7 mm to about 1.5 mm. The distance between two or more adjacent seals 30, 50, 60 may be uniform or may vary as desired.

In an exemplary embodiment, at least two adjacent seals 30, 50, 60 are closely positioned along passageway 12 such that they contact with and/or sealably engage with one another when a medical device is positioned in the longitudinal passageway 12. For example, when a medical device is positioned through seals 60, 50, and 30, inner surface 64 and/or a body region of supplemental membrane 60 proximate thereto may contact or sealably contact second seal 50. Similarly, at least inner surface 56 of second seal 50 may contact or sealably contact first seal 30. This configuration of the seals further ensures that the medical device forms a reliable seal with the introducer valve of the present invention.

The introducer valve, including the components of valve body 10 may be constructed from any suitable material, including but not limited to an elastomeric material, such as medical grade silicone, isoprene, or rubber. In one embodiment, the various components of valve body 10, specifically seals 30, 50 and 60, may be constructed from the same material. In an alternative embodiment, seals 30, 50 and 60 may be constructed from different materials of varying durometer hardness. Preferably, the material selected to construct first seal 30 has a lower durometer hardness than the material used to construct second seal 50 and/or one or more supplemental seals 60. In one embodiment, first seal 30 may be constructed from a material having a Shore A durometer of about 10 to about 50, preferably, from about 15 to about 40, and more preferably, from about 20 to about 30. Supplemental seal 60 may be constructed from a material having a Shore A durometer hardness of from about 15 to about 65, preferably, from about 20 to about 55, and more preferably, from about 25 to about 35. Additionally, second seal 50 may be constructed from a material having a Shore A durometer hardness of from about 10 to about 60, preferably, from about 15 to about 50, and more preferably, from about 25 to about 30.

In one embodiment, the introducer valve, including all the components of valve body 10, such as first seal 30, second seal 50 and one or more supplemental seals 60, may be constructed as an assembly formed from two or more component parts that are pressure fitted together by a hub 82 of introducer 80. Preferably, valve body 10 is constructed as an assembly of two that is designed for use with an introducer 80 configured as a splittable assembly. By virtue of the fact that each of the valve seals 30, 50, 60 of this embodiment are constructed as an assembly components, this structure reduces the distribution of resistive forces through the sealing element relative to seals of a valve body constructed as a unitary element. This is because only the resistive forces of half of valve body 10 are transferred to a particular location of a seal 30, 50, 60 when valve body 10 is constructed from two components, whereas when the valve body is constructed of a single component, the resistive forces of the entire valve body are transferred to a particular location of a seal.

Additionally, the two component assembly of valve body 10 of the present invention may facilitate splitting of a combination of valve body 10 and introducer 80 when it is desired to remove them at an appropriate time during or after a procedure. Specifically, valve body 10 may be located within introducer 80 with plane 20 aligned with score lines 98 of introducer 80 to facilitate the splitting process.

In another embodiment, valve body 10 may be constructed as an assembly of separate sections each including one or more of seals 30, 50, 60 that, when assembled, define a longitudinal passageway 12 for receiving a medical device in a similar manner to the embodiments depicted herein. These separate sections may have the same structure, configuration, material composition and arrangement as the seals 30, 50, 60 described above. As shown in FIG. 5(c), the valve assemblies may best be assembled using an adhesive applied in locations 160 and 165, said adhesive being strong enough to enhance the bond, but weak enough to allow for the valve to break apart during the splitting of the tearaway introducer. The adhesive can have similar mechanical properties as the valve material, such as modulus of elasticity and/or durometer or can be a harder more rigid material, or optionally a softer and more compliant material. It is preferred that the adhesive and valve material have similar mechanical properties since then the adhesive material will not interfere with the flexibility of the valve material in operation of the device. Examples include but are not limited to silicone, polyurethane, cyanoacrylic, epoxy, or other elastomer based adhesives.

Optionally, after applying adhesive in locations 160 and 165 the adhesive can flow across the entirety of surfaces 130 and 60 shown in FIGS. 5(a)-5(b). The valve can then be punctured with a sharp device such as trocar, pin, or needle creating a means for a medical device to pass through passage 12 while retaining a tight seal around said medical device.

Optionally, valve body 10 may further include a ledge 70 that couples valve 1 to a hub 82 of an introducer 80. As best shown in FIGS. 1(c)-1(d), ledge 70 has a cylindrical body with a T-shaped cross-section that is connected to and protrudes out from cylindrical wall 14. The lower edge 72 of ledge 70 is spaced apart from and forms a cylindrical groove 74 in conjunction with cylindrical wall 14. An upper edge 76 of ledge 70 is spaced apart and projects upwards from cylindrical wall 14, forming a cylindrical step. In one embodiment, ledge 70 may be located proximate to proximal end 16, preferably proximal to all of the seals.

Ledge 70 and valve body 10 may be coupled to an introducer 80, as shown in FIGS. 1(c)-1(d). Introducer 80 includes a hub 82 having a central cavity 84 for receiving valve assembly 1. As shown, hub 82 may include a base 86, within which valve body 10 is positioned, and a cap 88 that releasably attaches and secures valve body 10 to base 86. A ridge 90 positioned along an upper end of base 86 has a corresponding mating surface adapted to be releasably received in cylindrical groove 74. A corresponding projection 92 of cap 88 surrounds an upper surface 78 of ledge 70 and is snap fitted onto ledge 70, thereby providing a friction fit of ledge 70 between cap 88 and base 86. When positioned within hub 82, the distal end 18 of valve body 10 is vertically spaced apart from an introducer sheath 94 that is coupled to a distal end of hub 82 at a distal region of cavity 84.

When introducer 80 is configured as a splittable introducer assembly, two score lines 98 run along the length of hub 82, including base 86 and cap 88 for splitting introducer 80 into two components. A pair of handles 96 attached to opposing sides of hub 82 is positioned with one handle each on opposite sides of score lines 98 of introducer 80. Handles 96 provide a gripping surface for applying torque in order to initiate the splitting of introducer 80. Although score line 98 does not run along sheath 94, sheath 94 may be constructed from a tearable material or a material that promotes tearing along the length thereof.

The introducer valve of the present invention may be used with introducer 80 to facilitate insertion of a medical device into the body of a patient. When positioned within introducer 80, the introducer valve forms a self-sealing surface that prevents the passage of liquids or gases through introducer 80.

Upon making an incision, a surgeon inserts introducer 80 into a patient at a desired trajectory to position sheath 94 in a target subcutaneous location, such as a region adjacent to a blood vessel. Subsequently, a medical device, such as a guidewire or 30 gauge needle, may be inserted through the hub 82 of introducer 80 and valve body 10 to locate or fenestrate the blood vessel. Upon insertion into the longitudinal passageway 12 of valve body 10, the medical device passes through aperture 56 of second seal 50. The medical device may have such a small diameter that it does not directly engage the inner surface 54 of second seal 50 in which case a seal will be formed between first seal 30 and the medical device.

In embodiments where valve body 10 has one or more supplemental seals 60, the medical device also passes through each aperture 66 of the one or more supplemental seals 60. Depending upon the diameter of the medical device and size of the apertures 66, the medical device may or may not contact and form a seal with the inner surface 64 of the one or more supplemental seals 60 in which case a seal will be formed with either second seal 50 or first seal 30, depending upon the diameter of the medical device.

Subsequently, the medical device passes through first seal 30. When first seal 30 is formed as an assembly of two components, as shown, the medical device penetrates first seal 30 at mating surfaces 32 between these two components or, if valve body 10 is a single component, within a slit formed in valve body 10. In the embodiment shown in FIGS. 1(a)-1(b), 2(a)-2(b) 3(a)-3(b) and 5(a)-5(b), the tip of medical device is positioned such that it is aligned with and passes through the center of depression 36 and/or indentation 38. The mating surfaces 32 of the two components of first seal 30 deflect in a distal direction forming a seal with the medical device that is impervious to the passage of gases or liquids, such as blood if the medical device is suitably sized for first seal 30.

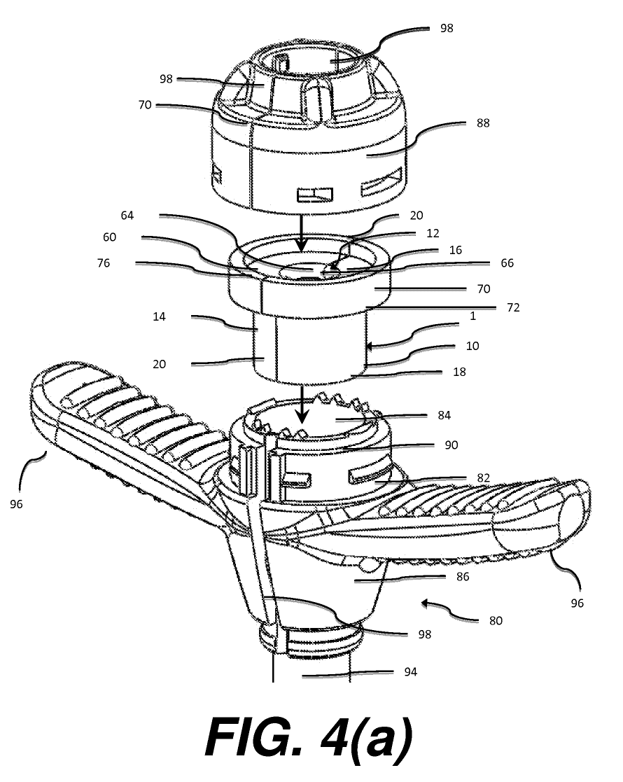

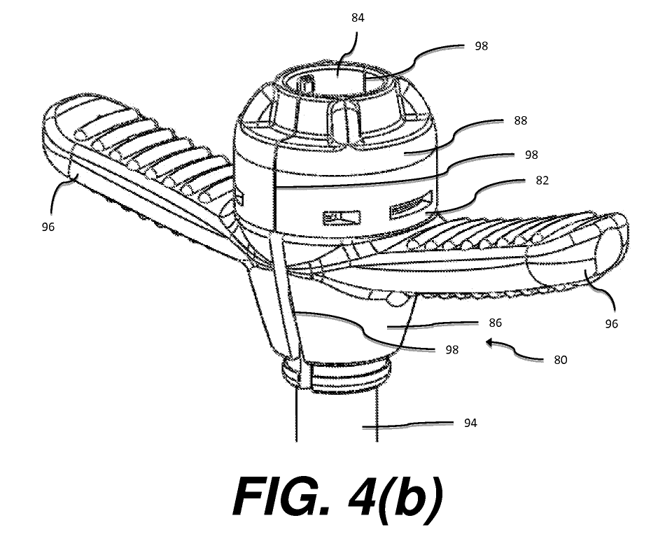

In the embodiment shown in FIGS. 4(a)-4(b), the medical device penetrates between the curved inner surface 144 of first seal 130. As the medical device is inserted through first seal 130, first seal 130 will deflect in a distal direction as the medical device is inserted through first seal 130. As the medical device penetrates first seal 130, it forms a seal with a location on curved inner surface 144 determined by the diameter of the medical device and hence the degree of distal deflection of first seal 130 that is impervious to the passage of gases or liquids, such as blood.

When first seal 30 is an integral one piece unit, the medical device penetrates one or more slits defined in first seal 30. The flaps of first seal 30 formed by one or more slits deflect in a distal direction and form a seal with the medical device that is impervious to the passage of gases or liquids, such as blood.

The surgeon may then manipulate the medical device to locate and/or fenestrate a target, such as a blood vessel. Upon withdrawing the medical device from introducer 80, the first seal 30 automatically resumes its original self-sealing orientation across longitudinal passageway 12 and thereby preventing the escape of gases or fluids through valve body 10.

The surgeon may then select another medical device having a larger diameter than the previous medical device, such as a catheter. The catheter may be inserted through valve body 10 and introducer 80 in the same manner as discussed above with respect to the previous medical device. Due to its larger size, the catheter will create a sealing engagement with one of apertures 56, 66 of second seal 50 and/or one or more supplemental seals 60, depending upon the size of apertures 56, 66 and the size of the catheter. This seal is formed in the same manner as described above in relation to first seal 30 shown in FIGS. 1(a)-3(b).

The surgeon may then use the catheter to perform a surgical procedure, such as introduce a material to the target site or drain a fluid, such as blood. Once the catheter is positioned introducer 80 and valve body 10 may be split apart at score line 98 and along plane 20 and removed from the catheter. Gripping handles 92, the surgeon may retract hub 82 and sheath 94. Upon applying torque to handles 92, the surgeon may initiate splitting of base 86 along score line 98 and continue to tear sheath 94 along its length. The tearaway introducer is assembled as in FIGS. 4(a) and 4(b) in such a way that the valve is retained in each half of the device as it is split. As in FIGS. 5(a)-5(c), adhesively bonded valve halves will separate as the assembly is torn along the break or score line.

The introducer valve of the present invention provides a number of advantages relative to prior art valves. The curved inner surface 144, flexural modulus and thickness of seals 30, 50, 60 enhance flexibility and the sealing capability of seals 30, 50, 60. Additionally, the arrangement of seals 30, 50, 60 in a tiered structure enables valve body 10 to seal with a wide range of sizes of medical devices. Moreover, by virtue of the fact that valve body is configured as an assembly of two halves as in FIGS. 4(a) and 4(b), this structure reduces the distribution of resistive forces throughout the seal that would otherwise substantially inhibit the insertion of a medical device and facilitates the splitting of valve body 10 for removal from the medical device.

The introducer valve of the present invention may be used for a wide variety of applications. While the introducer valve may be particularly suitable for use in forming a hemostatic seal, it may also be incorporated in introducers designed for non-vascular procedures where there is a desire to prevent leakage of fluids and/or reduce exposure to air-borne pathogenic organisms. For example, the introducer valve may be used to facilitate insertion of any medical device into any bodily cavity, including any subcutaneous cavity. In one embodiment, the introducer valve can be placed in an introducer for use in minimally invasive neurological procedures to limit contact of the cerebral spinal fluid with ambient air. In another embodiment, it may be positioned within a trocar for use in a laparoscopic procedure. Another possible application would be the use of the introducer valve in urological procedure where the valve could help prevent the introduction of pathogenic organisms into the urinary tract.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. An introducer valve comprising:
a valve body defining a longitudinal passageway for receiving a medical device;
a first sealing element forming a seal across the longitudinal passageway; and
a second sealing element spaced apart from the first sealing element, wherein the second sealing element comprises a curved inner surface defining a first aperture through the second seal.

2. The introducer valve of claim 1, wherein the inner surface has a curved configuration.

3. The introducer valve of claim 2, wherein the inner surface is formed by a semicircle, ellipse, cone or parabola.

4. The introducer valve of claim 1, wherein the first sealing element has a lower durometer hardness than the second sealing element.

5. The introducer valve of claim 1, wherein the first sealing element has a greater thickness than the second sealing element.

6. The introducer valve of claim 1, wherein the first sealing element includes a depression and/or indentation in its upper surface.

7. The introducer valve of claim 1, wherein the first aperture has a diameter of about 0.25 mm to about 4 mm.

8. The introducer valve of claim 1, wherein the first aperture has a diameter equal to or less than the maximum diameter of an insertable medical device by about 5% to about 60%.

9. The introducer valve of claim 1, further comprising a third sealing element, wherein the third sealing element has a semi-toroidal shape providing a curved inner surface defining a second aperture through the third sealing element.

10. The introducer valve of claim 9, wherein the second aperture is larger than the first aperture.

11. The introducer valve of claim 9, wherein the second aperture has a diameter of about 1.5 mm to about 9 mm.

12. The introducer valve of claim 1, wherein the valve body is formed by two independent parts that are pressure fitted together.

13. The introducer valve of claim 1, wherein the valve body is formed by two independent parts that are adhesively bonded together.

14. The introducer valve of claim 13, wherein the adhesively bonded valve body is adapted to be split by a secondary operation creating a passageway through the valve body.

15. An introducer valve comprising:
a valve body defining a longitudinal passageway for receiving a medical device;
a first sealing element forming a seal across the longitudinal passageway; and
a second sealing element spaced apart from the first sealing element, wherein the second sealing element comprises an inner surface defining a first aperture through the second sealing element, and wherein the valve body is formed from two independent parts that are pressure fitted or adhesively bonded together.

16. The introducer valve of claim 15, wherein the second sealing element has a lower flexural modulus than the first sealing element.

17. The introducer valve of claim 15, further comprising a third sealing element, wherein the third sealing element comprises an inner surface defining a second aperture through the third sealing element and wherein the third sealing element has a larger aperture than the second sealing element.

18. The introducer valve of claim 15, wherein the inner surface is curved.

19. The introducer valve of claim 15, wherein the inner surface is formed by a semi-circle, ellipse, cone or parabola.

20. The introducer valve of claim 15, wherein the first sealing element includes a depression and/or indentation in its upper surface.

21. The introducer valve of claim 15, wherein the valve body includes structure for coupling the valve body to an introducer.

22. The introducer valve of claim 15, wherein the valve body is formed by two independent parts that are adhesively bonded together.

23. The introducer valve of claim 22, wherein the adhesively bonded valve body is adapted to be split by a secondary operation creating a passageway through the valve body.

24. An introducer valve comprising:
a longitudinal passageway for receiving a medical device;
a first sealing element forming a seal across the longitudinal passageway;
a semi-toroidal second sealing element spaced apart from the first sealing element,
wherein the semi-toroidal second sealing element defines a first aperture; and
a semi-toroidal third sealing element positioned between the first sealing element and the semi-toroidal second sealing element, wherein the semi-toroidal third sealing element defines a second aperture that is larger than the first aperture.

25. An introducer valve comprising:
a longitudinal passageway for receiving a medical device;
a first sealing element forming a seal across the longitudinal passageway;
a semi-toroidal second sealing element spaced apart from the first sealing element,
wherein the semi-toroidal second sealing element defines a first, conically shaped aperture; and
wherein the semi-toroidal third sealing element defines a second aperture that is larger than the first aperture.
